(19) United States
(12) Patent Application Publication
(10) Pub. No.: US 2026/0207200 A1
(43) Pub. Date: Jul. 23, 2026

(54) MEDICAL APPARATUS FOR REGULATING ENDOLUMINAL FLOW AND RELATED METHODS

(71) Applicant: Bard Peripheral Vascular, Inc., Franklin Lakes, NJ (US)

(72) Inventor: Zachary Willis, Mesa, AZ (US)

(21) Appl. No.: 19/030,030

(22) Filed: Jan. 17, 2025

(51) Int. Cl. A61B 17/12; A61B 17/00

(52) U.S. Cl. CPC A61B 17/12036; A61B 17/12168; A61B 2017/00367; A61B 2017/00477; A61B 2017/1205

(57) ABSTRACT

A medical apparatus for partially occluding a body lumen includes a stent having a collapsed and an expanded configuration. A restrictor in the form of at least one cord is adapted for applying a tension to reduce a diameter of at least a portion of the stent in the expanded configuration, such as by being interwoven with a reticulated body of the stent. A retainer may retain the cord in place once the tension is applied to reduce the diameter of the portion of the stent. A cutter may also be provided for cutting the cord. More than one restrictor may be provided for restricting the diameter of different portions of the stent in order to create an internal passage with a complex geometry.

### BACKGROUND

Stents are often used in endoluminal applications, particularly in the vasculature, in order to restore patency or perform other functions. The ability to deploy the stent in a particular configuration to achieve a desired function is of considerable importance. This is particularly true when the goal is regulating fluid flow in the associated lumen and, thus, function as a valve or flow reducer, since the relative size, geometry and positioning of the stent are paramount considerations to achieve suitable flow control in the course of treating a particular medical condition.

To achieve the particular treatment objective at issue, the stent should also be able to undergo easy and quick modification in situ. This flexibility in use allows for the stent to accommodate placement in any of a variety of locations of varying size and shape in a body lumen, such as to achieve a particular level of flow regulation or perform other functions. Another consideration is to ensure that upon placement, the stent remains secure and stable in the vessel, yet the arrangement such that removal or re-positioning may be done in an efficient and effective manner when intended (and not otherwise).

Accordingly, a need is identified for a stent that may be reliably deployed in a particular configuration endoluminally and adapted to regulate fluid flow in the associated lumen in which it is located. The geometry of the stent would be able to undergo easy and quick modification in situ in order to accommodate placement in any of a variety of locations of varying size and shape in a body lumen. The stent would also be arranged to ensure that, once deployed, it remains secure and stable, yet subject to removal when intended.

### SUMMARY

An object of the disclosure is to provide a medical apparatus including an expandable stent that may be deployed in a particular configuration to serve as a regulator, valve or reducer for controlling fluid flow in the associated lumen. By using an inflatable balloon having portions with different compliance, the geometry of the stent would be able to undergo easy and quick modification in situ to accommodate placement in any of a variety of locations of varying size and shape in a body lumen, and provide a particular degree of flow regulation. The stent would also be arranged to ensure that, once deployed, the placement remains secure and stable, yet subject to removal when intended.

In one aspect, a medical apparatus for partially occluding a body lumen, comprises a stent having a reticulated body with a plurality of openings, the stent having a collapsed and an expanded configuration. At least one cord passes through the plurality of openings, the at least one cord adapted for applying a tension to reduce a diameter of at least a portion of the stent in the expanded configuration.

In one version, the reticulated body of the stent comprises a plurality of struts that are capable of relative sliding movement. The at least one cord may be interwoven through the plurality of openings. In one particular embodiment, the at least one cord is a first cord, and further including a second cord passing through a different plurality of openings in the reticulated body of the stent, such as by being interwoven.

In one aspect of the disclosure, a medical apparatus for partially occluding a body lumen is provided. The apparatus includes a stent having a collapsed and an expanded configuration. At least one cord is adapted for applying a tension to reduce a diameter of at least a portion of the stent in the expanded configuration. A retainer is provided for retaining the cord in place once the tension is applied to reduce the diameter of the portion of the stent.

In one embodiment, the retainer comprises a stopper on the cord, and in another, the retainer comprises a knot on the cord. The retainer in other embodiments comprises a housing including a passage for receiving the cord, the housing including at least one ball or a pivoting member for engaging the cord within the housing. The housing may be attached to the stent.

The at least one ball may comprise a pair of balls for engaging the housing when tension is applied to the cord. The housing may include includes a first recess for retaining the at least one ball in engagement with the cord when tension is applied to the cord and a second recess for releasing the at least one ball from engagement with the cord when tension is released therefrom.

The apparatus may further include a cutter for cutting the cord. The cutter may comprise part of a catheter for delivering the stent, and may have a blade for cutting the cord. The blade may be located along a channel for receiving a portion of the cord, such that rotation of the catheter moves the cord through the channel into engagement with the blade.

In any embodiment, the at least one cord may be adapted for applying tension for reducing the diameter of the stent at two different portions thereof. The at least one cord may comprise a first cord, and the apparatus may further include a second cord for applying a second tension to reduce a second diameter of a second portion of the stent.

The apparatus may also include a handle including a roller or a slider for applying tension to the cord. A conduit may also be provided for receiving the cord. The conduit may be on or in the stent.

A further aspect of the disclosure relates to a medical apparatus for partially occluding a body lumen. The apparatus includes a stent having a collapsed and an expanded configuration. The apparatus further includes at least one cord adapted for applying a tension to reduce a diameter of at least a portion of the stent in the expanded configuration, and a cutter for cutting the cord.

In one embodiment, the cutter is part of a catheter for delivering the stent. The cutter may include a blade for cutting the cord. The blade may be located along a channel for receiving a portion of the cord, whereby rotation of the catheter moves the cord through the channel into engagement with the blade.

The apparatus may further include a retainer for retaining the cord in place once the tension is applied to reduce the diameter of the portion of the stent. The retainer may comprise a stopper on the cord or a knot on the cord. The retainer may further comprise a housing including a passage for receiving the cord, the housing including at least one ball for engaging the cord within the housing, which may be attached to the stent. The at least one ball may comprise a pair of balls for engaging the housing when tension is applied to the cord. The housing may include a first recess for retaining the at least one ball in engagement with the cord when tension is applied to the cord and a second recess for releasing the at least one ball from engagement with the cord when tension is released therefrom.

The at least one cord may be adapted for applying tension for reducing the diameter of the stent at two different portions thereof. In one exemplary embodiment, the at least one cord is a first cord, and further including a second cord for applying a second tension to reduce a second diameter of a second portion of the stent.

The apparatus may further include a handle including a roller or a slider for applying tension to the cord. The apparatus may further include a conduit for receiving the cord, which may be on or in the stent.

A further aspect of the disclosure pertains to a medical apparatus for partially occluding a body lumen. The apparatus comprises a stent having a collapsed and an expanded configuration. The apparatus includes a first restrictor adapted for applying a first tension to reduce a first diameter of a first portion of the stent in the expanded configuration. The apparatus further includes a second restrictor adapted for applying a second tension to reduce a second diameter of a second portion of the stent in the expanded configuration.

In one example, the stent comprises a plurality of slidably engaged struts. In such case, the first restrictor comprises a first cord woven into the plurality of the struts of the first portion. Likewise, the second restrictor comprises a second cord woven into the plurality of struts at the second portion.

According to a further aspect of the disclosure, a method for partially occluding a body lumen includes expanding a stent in the body lumen, and independently reducing the diameter of first and second portions of the expanded stent. The reducing step may comprise comprises applying tension to at least one cord connected to the stent. In one particular version, the reducing step comprises applying tension to a first cord connected to the first portion of the stent and a second cord connected to the second portion of the stent.

### BRIEF DESCRIPTION OF THE DRAWINGS

The above and further advantages of the disclosure may be better understood by referring to the following description in conjunction with the accompanying drawings in which:

FIG. 1 is a perspective view of a balloon catheter.

FIG. 2 is a cutaway rear perspective view of the balloon portion of the balloon catheter of FIG. 1.

FIG. 2A is a cross-sectional view taken along line 2A-2A of FIG. 2.

FIG. 3 is an alternative embodiment of a balloon catheter.

FIGS. 4 and 4A are side views of an apparatus for use in regulating endoluminal flow according to one aspect of this disclosure.

FIG. 4B is an end view of the stent illustrating the interwoven cord.

FIGS. 5, 6, and 7 are schematic progressive views illustrating the functioning of the apparatus according to one aspect of the disclosure.

FIGS. 8 and 9 illustrate a handle forming part of the apparatus.

FIGS. 10, 10A, and 10B illustrate one version of a retainer for use in connection with the apparatus according to one aspect of the disclosure.

FIGS. 11, 12, 13, 14, and 14A illustrate additional versions of retainers.

FIGS. 15, 15A, and 15B illustrate a cutter for use in connection with the apparatus according to one aspect of the disclosure.

FIGS. 16, 17, and 18 illustrate an apparatus for use in regulating endoluminal flow according to another aspect of this disclosure.

The dimensions of some of the elements in the drawings may be exaggerated relative to other elements for clarity or several physical components may be included in one or element. Further, sometimes reference numerals may be repeated among the drawings to indicate corresponding or analogous elements. Moreover, some of the items depicted in the drawings may be combined into a single function.

### DETAILED DESCRIPTION

In the following detailed description, numerous specific details are set forth to provide a thorough understanding of the disclosure in relation to various inventions. The disclosed embodiments may be practiced without these specific details. In other instances, well-known methods, procedures, components, or structures may not have been described in detail so as not to obscure the present invention.

The principles and operation of the apparatus and methods of the disclosure may be better understood with reference to the drawings and accompanying descriptions. The invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein are for the purpose of description and should not be regarded as limiting.

Certain features of the invention that are, for clarity, described in the context of separate embodiments, may also be provided in combination in a single embodiment. Conversely, various features of the invention that are, for brevity, described in the context of a single embodiment, may also be provided separately or in any suitable sub-combination. The description provided below and in regard to the figures applies to all embodiments unless noted otherwise, and features common to each embodiment are similarly shown and numbered to the greatest extent possible to lend clarity, but should not be considered limiting in any respect.

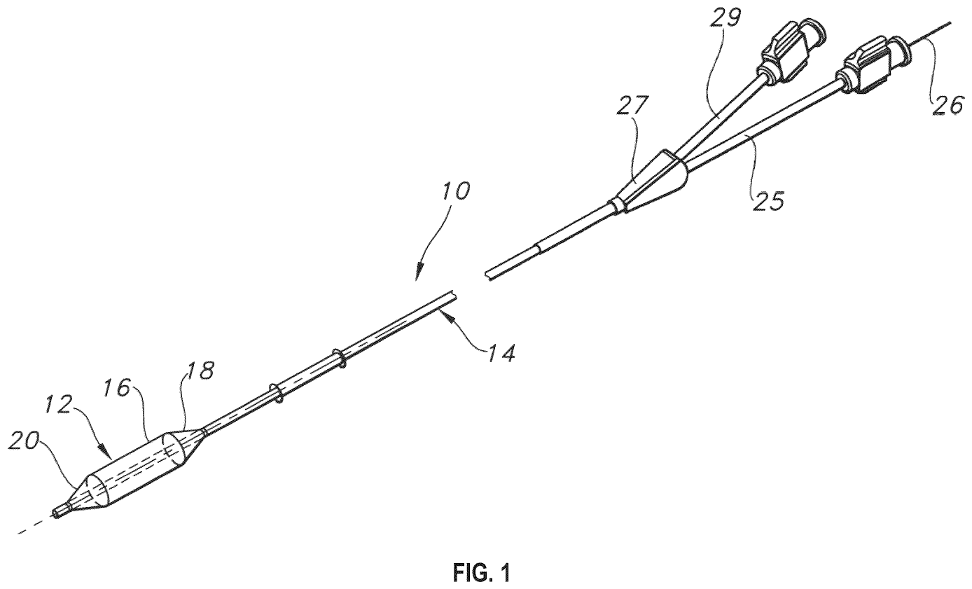

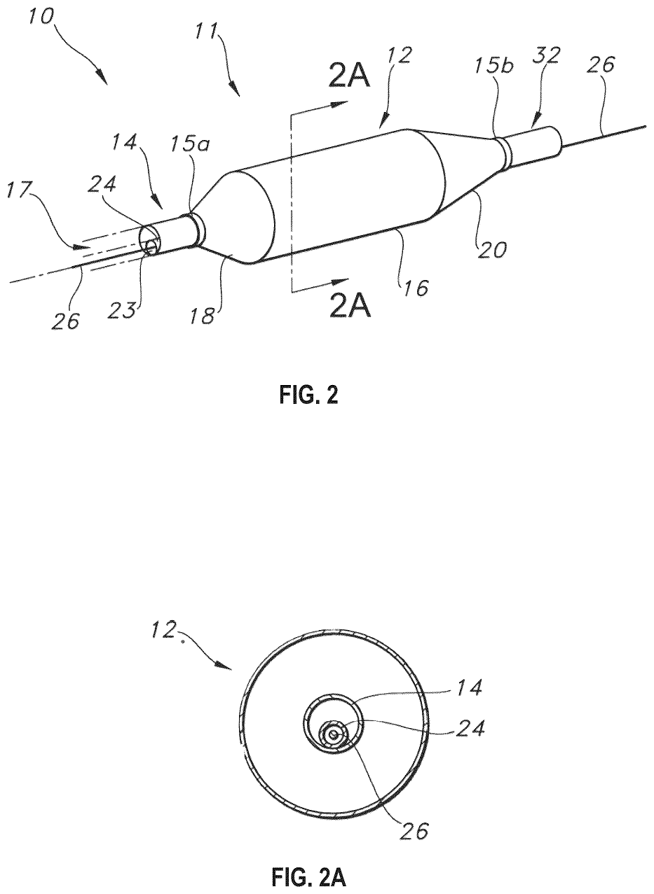

With reference to FIGS. 1, 2, and 2A, provided is a catheter 10 having a distal end portion 11 with a balloon 12 mounted on a catheter shaft 14. The balloon 12 has an intermediate portion 16, or “barrel,” and end portions 18, 20. In one embodiment, the end portions 18, 20 reduce in diameter to join the intermediate portion 16 to the catheter shaft 14 (and thus portions 18, 20 are generally termed cones or cone portions). The balloon 12 is sealed at balloon ends (proximal end 15a and distal end 15b) on the cone portions 18, 20 to allow the inflation of the balloon 12 via one or more inflation lumens 17 extending within catheter shaft 14 and communicating with the interior of the balloon 12.

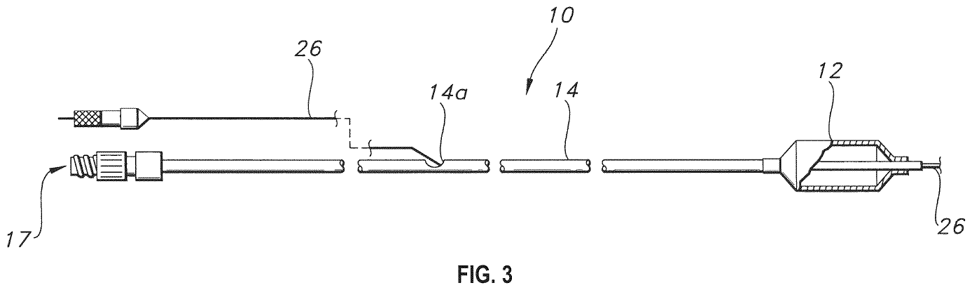

The catheter shaft 14 may also include an elongated, tubular shaft 24 forming a guidewire lumen 23 that directs the guidewire 26 through the catheter 10, and along the distal end portion of which the balloon 12 may be located. As illustrated in FIG. 2, this guidewire 26 may extend through the proximal end portion of the catheter 10 and a first port 25 of a connector or hub 27 into the lumen 23 to achieve an “over the wire” (OTW) arrangement, but could also be provided in a “rapid exchange” (RX) configuration (in which the guidewire 26 exits a lateral opening 14a closer to the distal end; see FIG. 3) or else is fed through the tip at a passage distally of the balloon 12 (“short” RX; not shown). A second port 29 may also be associated with catheter 10, such as by way of connector 27, for introducing a fluid (e.g., saline, a contrast agent, or both) into the interior compartment of the balloon 12 via the inflation lumen 17.

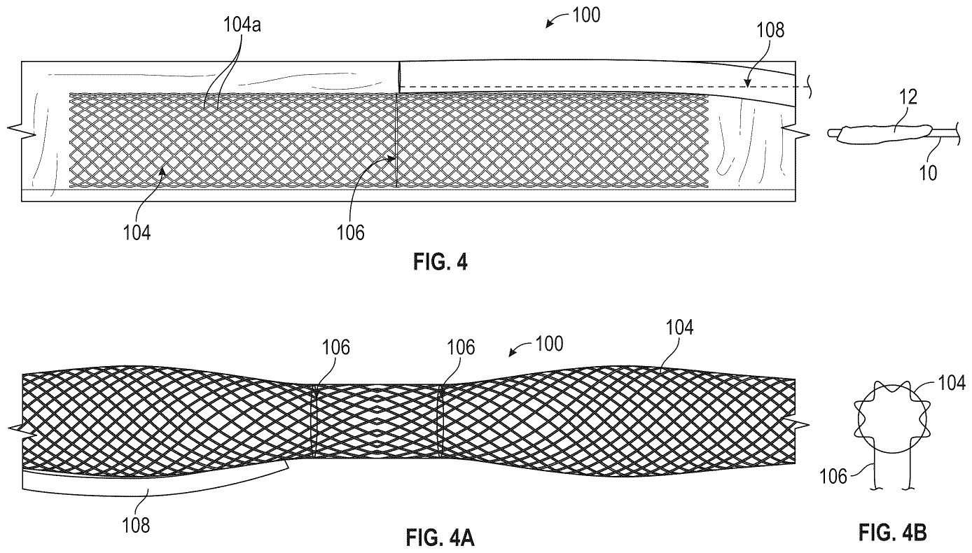

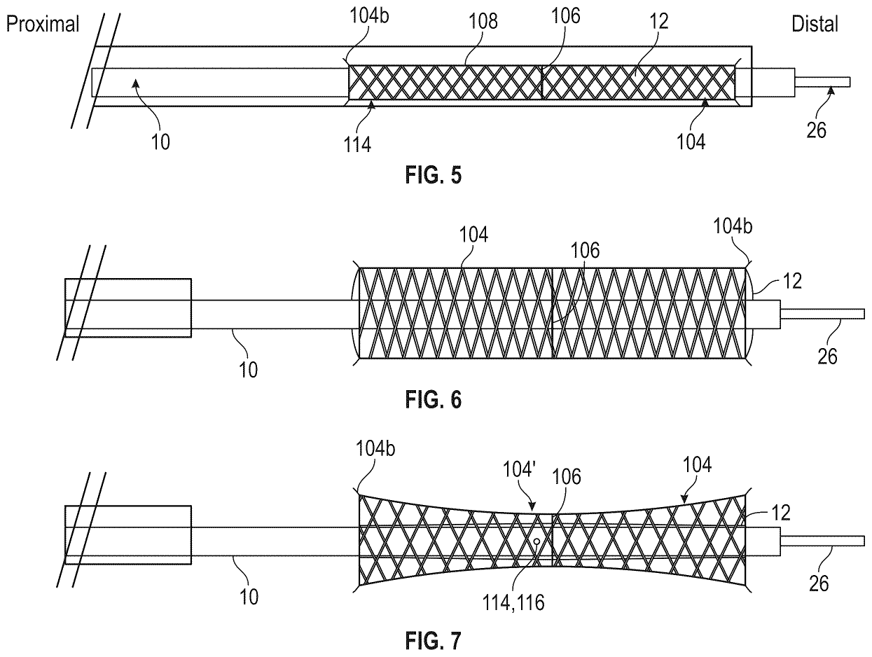

According to a first aspect of the disclosure, an apparatus 100 is provided that may be adapted in situ to control or regulate endoluminal fluid flow in a vessel of a body, such as for example a human blood vessel (or, stated another way, the apparatus is a “flow reducer”). In one embodiment, as shown in FIGS. 4 and 4A, the apparatus 100 involves the temporary use of an expandable structure, such as an inflatable balloon 12, in order to expand an expandable stent 104 into a particular configuration for regulating flow within a body lumen. As indicated in FIGS. 5, 6, and 7, the balloon 12 may be associated with a support, such as the catheter 10 described above, for intravascular positioning and inflation, as outlined in elsewhere in this description. In other versions, the stent 104 may be self-expandable, such as by being formed of a shape memory material, such as Nitinol.

The expandable stent 104 may be sized for positioning over the unexpanded or folded balloon 12 in a collapsed configuration (see FIG. 5). This stent 104 may comprise a reticulated body having a mesh, screen, or cage like structure with a plurality of openings. The corresponding structure of the body forming the stent 104 may include a plurality of struts 104a, which in the illustrated embodiment are helically arranged and woven or braided together. Despite being interwoven, adjacent struts 104a are not directly connected to each other, such as by welds or the like, and thus are capable of relative movement (e.g., by sliding), such as is the situation in a conventional “Chinese finger trap” device. The struts 104a may comprise metal, such as stainless steel, or other inelastic resilient materials, including possibly a shape memory material (e.g., Nitinol). The stent 104 may further include anchors 104b at or near the ends to prevent migration once expanded in a vessel.

The stent 104 may also be adapted to change shape, both in a radial direction and in a longitudinal direction, as a result of an externally applied force. As indicated in FIGS. 4 and 4A, this may achieved using a restrictor, which may comprise a tension or pull cord 106. A distal end portion of this cord 106 may be woven circumferentially within the struts 104a of the stent 104. The cord 106 at a proximal end portion may extend from the stent 104 to a location outside of the body by way of a conduit 108, which may be external to or internal to the stent 104. The distal end of the cord 106 may be fastened to the stent 104, such as by welding, adhesive, mechanical fastener, or the like, or may form an end loop and return back in a parallel path to adjacent to the proximal end portion. The stent 104 may also include features that guide the cord 106 circumferentially, such as an eyelet, tract, or the like (not shown).

The weaving of the distal portion of the cord 106 through the struts 104a may be done purely circumferentially, as shown in FIG. 4B. Alternatively, the weaving of the cord 106 through the struts 104a may be done at an angle, or in a combination of angles or directions, to achieve non-circumferential compression and in some circumstances provide unique internal shapes as a result of the differential narrowing achieved. In any case, when tension is applied to the cord 106, a reduction in the diameter of a corresponding portion of the stent 104 is created, as indicated in FIG. 4A (which shows the reduction in diameter by a cord 106 provided at different locations, which is considered optional, and as further outlined in the following description).

An exemplary sequence of operation is now described with reference to FIGS. 5, 6, and 7. Specifically, FIG. 5 illustrates that the stent 104 may be delivered endoluminally to a desired site via a catheter 10 including an expansion device, such as balloon 12. The delivery may involve the use of a guidewire 26 and an external deployment sheath 114, per the conventional technique.

Once positioned, the balloon 12 may be used to expand the stent 104 to a generally consistent, tubular configuration, or alternatively, the stent may self-expand upon retraction of the deployment sheath 114, as shown in FIGS. 6 and 7. Tension may then be applied to the pull cord 106 to reduce the diameter of, or “cinch” the stent 104 along a corresponding portion, such as the middle (see cinched portion 104′). The result is an hourglass-shaped stent 104, as shown in FIG. 7. The catheter 10 with the unexpanded or collapsed balloon 12 may then be withdrawn with the stent 104 remaining in place.

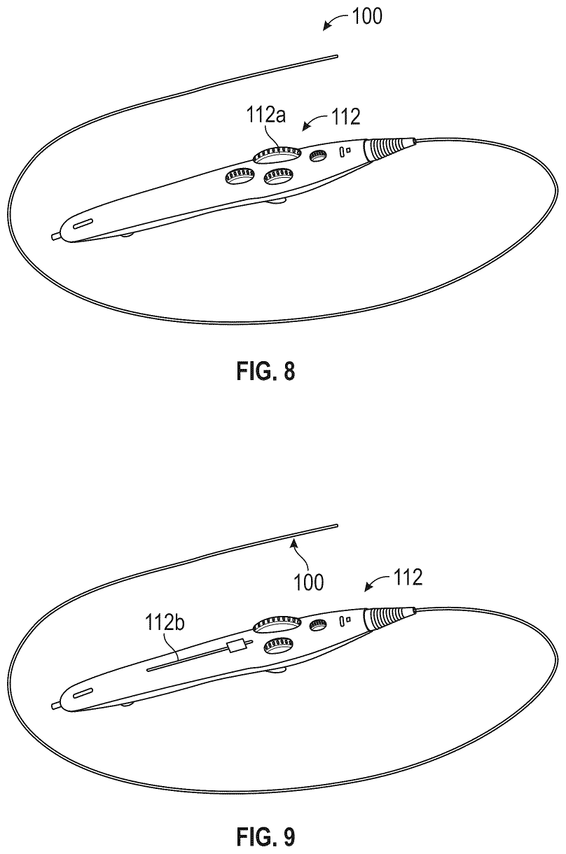

In the simplest version, tension may be applied to the cord 106 by simply pulling on it manually in a proximal direction (that is, away from the direction in which the stent was introduced into the body vessel). In another version, the applied tension may be controlled by use of an external puller associated with a handle 112, as shown in FIG. 8. The puller may comprise, for example, a roller, such as a thumbwheel 112a, which may be connected to a proximal end of the cord 106 and serve to pull it longitudinally when rotated (with the distal end of the cord 106 remaining fixed, such as to the stent 104 or to the handle 112). Alternatively, as shown in FIG. 9, a slider 112b may be provided for applying tension to the free proximal end of the cord 106, with the distal end remaining fixed. The amount of rotation or sliding achieved may be mechanically controlled, such as by a ratchet, to provide for a predetermined amount of tension and thus reduction in the diameter of the corresponding portion of the stent 104 as a result.

In order to retain the applied tension on the cord 106, it may be desirable to secure it at least partially to the stent 104. This may be achieved in one example by a knot 116 applied to the cord 106 to serve as a retainer. This knot 116 may comprise, for example, a cinch knot (e.g., a taut-line hitch), which may be tied into the cord 106 during manufacturing. Hence, applying tension to the cord 106 serves to cinch the corresponding portion of the stent 104 via the action of the knot 116.

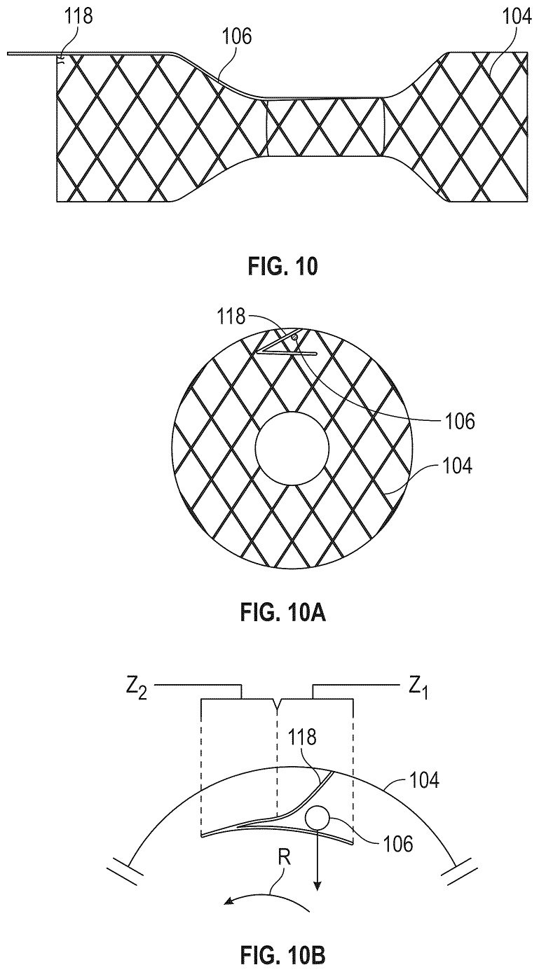

With reference to FIGS. 10, 10A, and 10B, a retainer may also be integrally formed with the stent 104. In one example, this retainer takes the form of a clip 118, which may be provided on or in the stent 104, in order to connect with a portion of the cord 106 proximal to the location where it is interwoven. In the illustrated example, the clip 118 is provided at a proximal end of the stent 104, and includes an opening facing in a circumferential direction forming a low compression zone, Z1, followed by a high compression zone, Z2, created by tapering of the clip 118. Thus, the stent 104 may be rotated to capture a corresponding portion of the cord 106 in the clip 118 and, in particular, the high compression zone thereof. This serves to fix the tension applied by the upstream portion of the cord 106 to the stent 104, and thus set the reduced diameter portion unless and until the tension is released (such as by reversing the rotation and releasing the applied tension-note action arrow R). The clip 118 could also serve as a combined retainer and cutter by providing a portion of it with a blade for cutting the cord 106.

Another possible version of a retainer is shown in FIG. 11. In this version, the retainer includes a housing 120 for receiving a portion of the tension cord 106. An internal passage 120a of the housing includes a pair of balls 122, which may have surface texture to grip an adjacent portion of the cord 106. The passage 120a is tapered in the proximal direction such that, when tension is applied to the cord 106, the balls 122 are urged proximally to a location where the adjacent portion of the cord 106 is captured, thus fixing the tension on an upstream portion thereof. The balls 122 are able to fully lock into place after releasing tension at the cord 106 (i.e., the stent 104 tries to expand and retracts cord 106 into locked position). At that time, the cord 106 may be cut and removed at final placement.

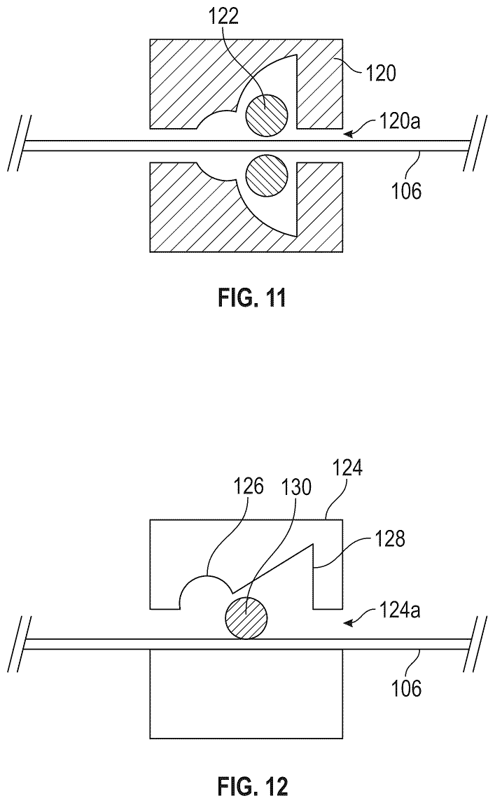

A further version of a retainer is shown in FIG. 12. In this version, a housing 124 including a passage 124a for a portion of the cord 106 is provided, which passage includes proximal and distal recesses 126, 128 of different sizes. In particular, the proximal recess 126 is smaller than the distal recess 128, such that the former captures a textured ball 130 in place, and the latter allows it to move away from the cord 106 in the passage 124a. Thus, applying tension to the cord 106 moves the ball 130 into the smaller proximal recess 126, securing the cord in place with the applied upstream tension.

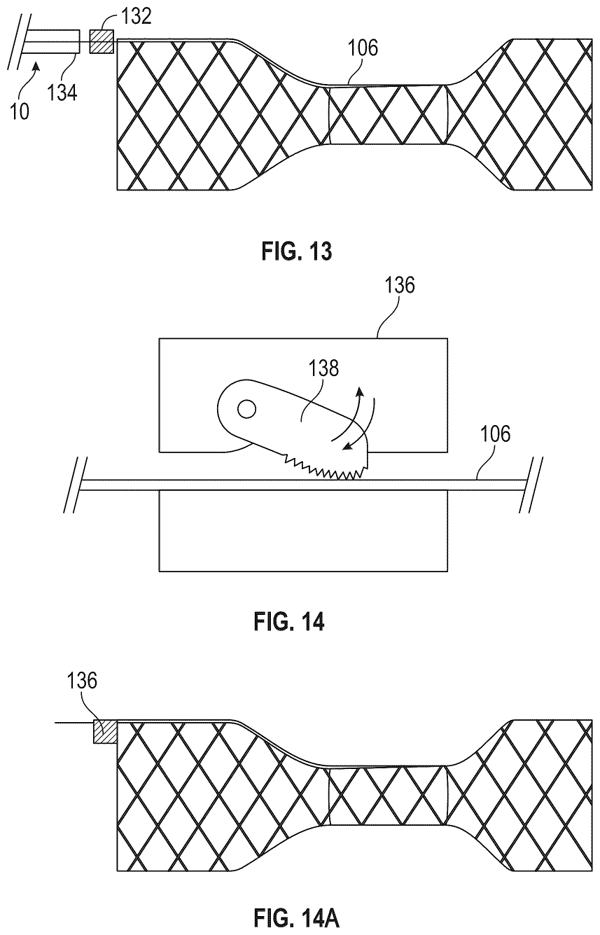

Still another version of a retainer is shown in FIG. 13, in which the retention function is achieved by positioning a stopper 132 along the cord 106 such as adjacent to the end of the stent 104 to retain in in place once the tension is applied. The stopper 132 may be advanced into position by a plunger 134 associated with the catheter 10.

In yet another version, as shown in FIGS. 14 and 14A, a housing 136 is provided for receiving a portion of the cord 106. The housing includes a pivotally mounted member 138 adapted to engage the cord 106 in a depending position, such as by way of teeth or like projections. Pulling the cord 106 proximally thus is permitted, and once tension is released, back pressure from the constricted stent pulls the cord 106 in the opposite direction, thus causing the member 138 to fix the relative position.

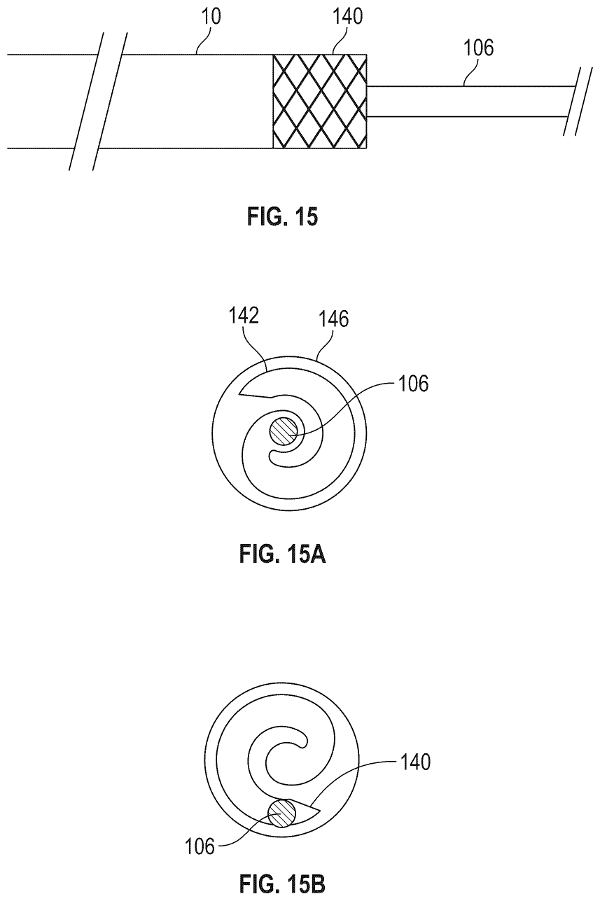

Once the cord 106 is properly tensioned and fixed in position relative to the stent 104, it may be desirable to sever the cord 106 proximal to the stent 104. This may be achieved by providing a cutter 140 on the catheter 10. In one example, as shown in FIGS. 15, 15A, and 15B, the cutter 140 may comprise a blade 142, which may be located at one end of a spiral channel 146, the center of which may include the tension cord 106 (such as by being positioned therein during manufacturing). Once the desired tension is applied and the cord 106 is captured using a retainer, the catheter may then be rotated to move a corresponding portion of the cord into contact with the blade 142, thus severing the connection and allowing for removal of the catheter while the custom-modified stent remains in place.

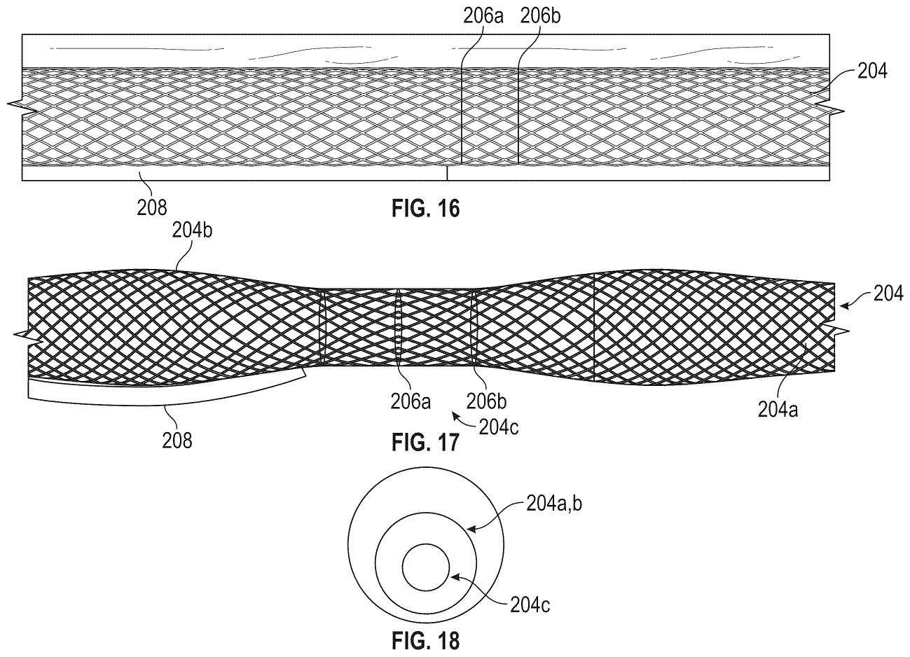

A further embodiment of the apparatus 200 is shown with reference to FIGS. 16, 17, and 18. In this version, the stent 204 is provided with more than one restrictor, such as more than one tension or pull cord. For example, as illustrated, one pull cord 206a may be located at a proximal location and one pull cord 206b may be located at a distal location. As above, the cords 206a, 206b may be interwoven with the struts 204a of the stent 204, which may be of similar configuration to struts 104a of stent 104. The cords 206a, 206b may extend through a conduit 208 associated with the stent 104 (internally or externally, as previously noted).

By applying tension to both cords 206a, 206b, such as by using the above-described handle 112, which may include independent tensioners for each cord, the stent 204 may thus be modified to include a narrower intermediate portion 204c bounded by larger diameter end portions 204a, 204b, thus provided a dog-bone configuration, with each end having a funnel-like shape bounding a narrow middle passage.

The relative diameters achieved may be altered by applying different amounts of tension to the cords 206a, 206b, which may be secured in place and cut as described herein, or the cords may be similarly tensioned to provide the intermediate portion 204c with a generally constant or consistent diameter. As can be appreciated, while two cords 206a, 206b are shown, any number may be used, and the cord(s) may be positioned anywhere along the stent 204, including possibly at the ends thereof.

Various additional modifications may be made to the apparatus according to this disclosure. For example, a lining may be applied to the stent 104, such as membrane. This lining may comprise, for example, a highly compressed PTFE material arranged in the form of a tube. Consequently, the lining may be expandable upon the application of internal pressure, such as by way of an expandable balloon, and caused to retain the expanded shape.

Summarizing, this disclosure may relate to any of the following concepts in any combination:
1. A medical apparatus for partially occluding a body lumen, comprising:
a stent having a reticulated body with a plurality of openings, the stent having a collapsed and an expanded configuration; and
at least one cord passing through the plurality of openings, the at least one cord adapted for applying a tension to reduce a diameter of at least a portion of the stent in the expanded configuration.
2. The medical apparatus of item 1, wherein the reticulated body of the stent comprises a plurality of struts that are capable of relative sliding movement.
3. The medical apparatus of item 1 or item 2, wherein the at least one cord is interwoven through the plurality of openings.
4. The medical apparatus of any of items 1-3, wherein the at least one cord is a first cord, and further including a second cord passing through a different plurality of openings in the reticulated body of the stent.
5. A medical apparatus for partially occluding a body lumen, comprising:
a stent having a collapsed and an expanded configuration;
at least one cord adapted for applying a tension to reduce a diameter of at least a portion of the stent in the expanded configuration; and
a retainer for retaining the cord in place once the tension is applied to reduce the diameter of the portion of the stent.
6. The medical apparatus of item 5, wherein the retainer comprises a stopper on the cord or a knot on the cord.
7. The medical apparatus of item 5 or item 6, wherein the retainer comprises a housing including a passage for receiving the cord, the housing including at least one ball for engaging the cord within the housing.
8. The medical apparatus of any of items 5-7, wherein the at least one ball comprises a pair of balls for engaging the housing when tension is applied to the cord.
9. The medical apparatus of any of items 5-8, wherein the housing includes a first recess for retaining the at least one ball in engagement with the cord when tension is applied to the cord and a second recess for releasing the at least one ball from engagement with the cord when tension is released therefrom.
10. The medical apparatus of any of items 5-9, wherein the retainer comprises a housing including a pivoting member for engaging the cord.
11. The medical apparatus of any of items 5-10, further including a cutter for cutting the cord.
12. The medical apparatus of any of items 5-11, wherein the at least one cord is adapted for applying tension for reducing the diameter of the stent at two different portions thereof.
13. The medical apparatus of any of items 5-12, wherein the at least one cord is a first cord, and further including a second cord for applying a second tension to reduce a second diameter of a second portion of the stent.
14. The medical apparatus of any of items 5-13, further including a handle including a roller or a slider for applying tension to the cord.
15. The medical apparatus of any of items 5-14, further including a conduit for receiving the cord, wherein the conduit is on or in the stent.
16. A medical apparatus for partially occluding a body lumen, comprising:
a stent having a collapsed and an expanded configuration;
at least one cord adapted for applying a tension to reduce a diameter of at least a portion of the stent in the expanded configuration; and
a cutter for cutting the cord.
17. The medical apparatus of item 16, wherein the cutter is part of a catheter for delivering the stent or is part of the stent.
18. The medical apparatus of item 16 or item 17, wherein the cutter comprises a blade located along a channel for receiving a portion of the cord, whereby rotation of the catheter moves the cord through the channel into engagement with the blade.
19. The medical apparatus of any of items 16-18, further including a retainer for retaining the cord in place once the tension is applied to reduce the diameter of the portion of the stent.
20. The medical apparatus of any of items 16-19, wherein the at least one cord is adapted for applying tension for reducing the diameter of the stent at two different portions thereof.
21. The medical apparatus of any of items 16-20, wherein the at least one cord is a first cord, and further including a second cord for applying a second tension to reduce a second diameter of a second portion of the stent.
22. The medical apparatus of any of items 16-21, further including a handle including a roller or a slider for applying tension to the cord.
23. The medical apparatus of any of items 16-22, further including a conduit for receiving the cord, wherein the conduit is on or in the stent.
24. A medical apparatus for partially occluding a body lumen, comprising:
a stent having a collapsed and an expanded configuration;
a first restrictor adapted for applying a first tension to reduce a first diameter of a first portion of the stent in the expanded configuration; and
a second restrictor adapted for applying a second tension to reduce a second diameter of a second portion of the stent in the expanded configuration.
25. The medical apparatus of item 24, wherein the stent comprises a plurality of slidably engaged struts, the first restrictor comprises a first cord woven into the plurality of the struts of the first portion and the second restrictor comprises a second cord woven into the plurality of struts at the second portion.
26. A method for partially occluding a body lumen, comprising:
expanding a stent in the body lumen; and
independently reducing the diameter of first and second portions of the expanded stent.
27. The method of item 26, wherein the reducing step comprises applying tension to at least one cord connected to the stent.
28. The method of item 26 or item 27, wherein the reducing step comprises applying tension to a first cord connected to the first portion of the stent and a second cord connected to the second portion of the stent.

As used herein, the following terms have the following meanings: “A”, “an”, and “the” as used herein refers to both singular and plural referents unless the context clearly dictates otherwise. By way of example, “a compartment” refers to one or more than one compartment. “About,” “substantially,” or “approximately,” as used herein referring to a measurable value, such as a parameter, an amount, a temporal duration, and the like, is meant to encompass variations of +/−20% or less, including +/−10% or less, +/−5% or less, +/−1% or less, and +/−0.1% or less of and from the specified value, in so far such variations are appropriate to perform in the disclosed invention. However, it is to be understood that the value to which such modifiers refer is itself also specifically disclosed. Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Although the invention has been described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

1. A medical apparatus for partially occluding a body lumen, comprising:
a stent having a reticulated body with a plurality of openings, the stent having a collapsed and an expanded configuration; and
at least one cord passing through the plurality of openings, the at least one cord adapted for applying a tension to reduce a diameter of at least a portion of the stent in the expanded configuration.

2. The medical apparatus of claim 1, wherein the reticulated body of the stent comprises a plurality of struts capable of relative sliding movement.

3. The medical apparatus of claim 1, wherein the at least one cord is interwoven through the plurality of openings.

4. The medical apparatus of claim 1, wherein the at least one cord is a first cord, and further including a second cord passing through a different plurality of openings in the reticulated body of the stent.

5. A medical apparatus for partially occluding a body lumen, comprising:
a stent having a collapsed and an expanded configuration;
at least one cord adapted for applying a tension to reduce a diameter of at least a portion of the stent in the expanded configuration; and
a retainer for retaining the cord in place once the tension is applied to reduce the diameter of the portion of the stent.

6. The medical apparatus of claim 5, wherein the retainer comprises a stopper on the cord or a knot on the cord.

7. The medical apparatus of claim 5, wherein the retainer comprises a housing including a passage for receiving the cord, the housing including at least one ball for engaging the cord within the housing.

8. The medical apparatus of claim 7, wherein the at least one ball comprises a pair of balls for engaging the housing when tension is applied to the cord.

9. The medical apparatus of claim 5, wherein the housing includes a first recess for retaining the at least one ball in engagement with the cord when tension is applied to the cord and a second recess for releasing the at least one ball from engagement with the cord when tension is released therefrom.

10. The medical apparatus of claim 5, wherein the retainer comprises a housing including a pivoting member for engaging the cord.

11. The medical apparatus of claim 5, further including a cutter for cutting the cord.

12. The medical apparatus of claim 5, further including a handle including a roller or a slider for applying tension to the cord.

13. The medical apparatus of claim 5, further including a conduit for receiving the cord, wherein the conduit is on or in the stent.

14. A medical apparatus for partially occluding a body lumen, comprising:
a stent having a collapsed and an expanded configuration;
at least one cord adapted for applying a tension to reduce a diameter of at least a portion of the stent in the expanded configuration; and
a cutter for cutting the cord.

15. The apparatus of claim 14, wherein the cutter is part of a catheter for delivering the stent or is part of the stent.

16. The apparatus of claim 14, wherein the cutter comprises a blade located along a channel for receiving a portion of the cord, whereby rotation of the catheter moves the cord through the channel into engagement with the blade.

17. The apparatus of claim 14, further including a retainer for retaining the cord in place once the tension is applied to reduce the diameter of the portion of the stent.

18. A medical apparatus for partially occluding a body lumen, comprising:
a stent having a collapsed and an expanded configuration;
a first restrictor adapted for applying a first tension to reduce a first diameter of a first portion of the stent in the expanded configuration; and
a second restrictor adapted for applying a second tension to reduce a second diameter of a second portion of the stent in the expanded configuration.

19. The medical apparatus of claim 18, wherein the stent comprises a plurality of slidably engaged struts, the first restrictor comprises a first cord woven into the plurality of the struts of the first portion and the second restrictor comprises a second cord woven into the plurality of struts at the second portion.
